(19) United States
(12) Patent Application Publication
(10) Pub. No.: US 2006/0189878 A1
(43) Pub. Date: Aug. 24, 2006

(54) Method to Automatically Generate 'Guided Meditation' Audio Data File

(76) Inventors: Hemant Joshi, Milpitas, CA (US); Jaya Joshi, Milpitas, CA (US)

Correspondence Address: HEMANT JOSHI, 1260 STARDUST WAY, MILPITAS, CA, 95035, US

(21) Appl. No.: 10/906,492

(22) Filed: Feb. 23, 2005

(51) Int. Cl. A61B 5/08

(52) U.S. Cl. 600/529

(57) ABSTRACT

An invention is disclosed for automatically generating the ‘Guided Meditation’ using the customer Preferences and Requirements. The customer Preferences and requirements if specified can be used to select meditation stages, language used for instructions, duration, and theme of meditation and ambience sounds for the entire ‘Guided Meditation’. The customer may specify the Preferences and Requirements over Internet, phone, email or postal mail and the automatically generated ‘Guided Meditation’ can be delivered as Internet download, as an email attachment or as audio CD or audiocassette by postal mail.

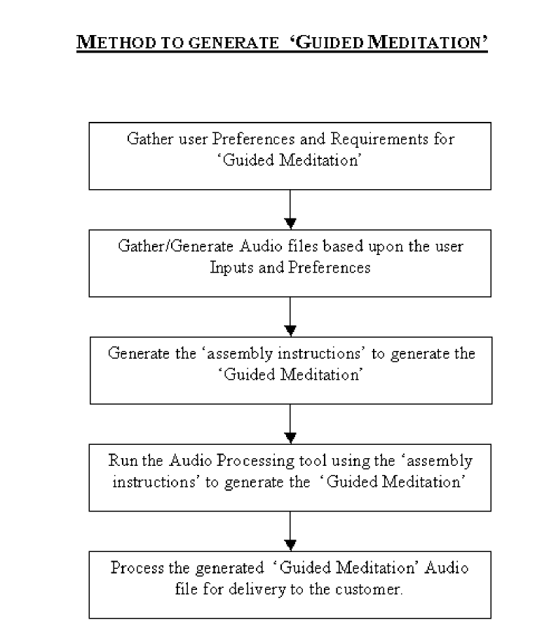

### FIELD OF INVENTION

The present invention relates generally to ‘Guided Meditation’ audio data file generation. It relates more specifically to methods, to automatically generate the ‘Guided Meditation’ audio data file based upon customer preferences and requirements.

### DESCRIPTION OF THE RELATED ART

‘Guided Meditation’ audio is generally composed of instructions and ambience sounds. Some times it is just composed with no instructions and sometime it contains only instructions. Generally, it is available in forms of audio CD, cassette or audio stream file over Internet. Currently the customer gets whatever is available and the customization to individual's preferences isn't available.

The ‘method’ or ‘invention’ uses the audio data stored as files, these files are written in prior art industry standard data formats. So in the entire document ‘audio data’, ‘audio files’ or ‘audio data files’ refer to the audio data written in these prior art industry standard data formats.

In this ‘invention’ prior art ‘audio processing tools’ are used to enable mixing various audio files in a defined sequence and generate one audio file containing complete ‘Guided Meditation’. These prior art ‘audio processing tools’ are readily available in the Market as commercial tools, shareware or freeware. These prior art ‘audio processing tools’ can also be programmed to detect certain patterns in the ‘audio data file’. These patterns can be sound of inhale and exhale if the ‘sound of breathing’ is recorded in the ‘audio data file’.

### SUMMARY OF THE INVENTION

Broadly speaking, the present invention satisfies the need of a customer to get the ‘Guided Meditation’ customized to fine details. It enables the customer to get the ‘Guided Meditation’ that fits the customer's personality, preferences and the defined requirements. This ‘method’ or ‘invention’ enables the customer to remotely and automatically generate the ‘Guided Meditation’ fitting all the preferences and the defined requirements. In this document an algorithm for creating ‘Guided Meditation’ audio file based upon customer preferences and defined requirements is described and it's preferred embodiments are discussed.

In one embodiment this invention enables the customer to specify all the preferences and requirements over the Internet and then either stream in or download the automatically generated ‘Guided Meditation’ in any of the desired/popular audio formats.

In yet another embodiment this invention enables the customer to provide the audio components for any or all of the phases of the ‘Guided Meditation’ or ambience in addition to preferences and requirements, which are accepted over the internet and then the ‘Guided Meditation’ is generated and provided to the user for download.

In yet another embodiment this invention enables the customer to provide the phases, their sequence, the ambience and audio components for any or all the phases and additional preferences and requirements, which are accepted over the Internet and then the ‘Guided Meditation’ is generated and provided to the user for download.

In yet another embodiment this invention enables the customer to specify all the preferences and requirements over the phone/email to the customer service representative, who in turn executes the ‘method’ to generate the ‘Guided Meditation’ in any of the desired/popular audio formats. This generated ‘Guided Meditation’ can then be shipped to the customer in form of an email, a CD or cassette or customer can directly download it over the Internet.

In yet another embodiment this invention enables the customer to provide audio components for any or all of the phases of the ‘Guided Meditation’ or ambience in addition to specify all the preferences and requirements over the phone/email to the customer service representative, who in turn executes the ‘method’ to generate the ‘Guided Meditation’ in any of the desired/popular audio formats. This generated ‘Guided Meditation’ can then be shipped to the customer in form of an email, a CD or cassette or customer can directly download it over the Internet.

In yet another embodiment this invention enables the customer to provide the phases, their sequence, the ambience and audio components for any or all the phases and additional preferences and requirements over the phone to the customer service representative, who in turn executes the ‘method’ to generate the ‘Guided Meditation’ in any of the desired/popular audio formats. This generated ‘Guided Meditation’ can then be shipped to the customer in form of an email, a CD or cassette or customer can directly download it over the Internet.

In yet another embodiment a special ‘Guided Meditation’ stage is generated that is based upon rhythmic breathing. This stage is generated using the prior art ‘audio processing software’ from the ‘sound of breathing’ audio data file, provided by the customer and recorded under different emotional states. This prior art software can analyze the ‘sound of breathing’ audio data file to distinguish between inhale and exhale patterns and period in between. This data for inhale and exhale pattern and rhythm can be used to generate one of the Meditation stages.

### DETAILED DESCRIPTION OF THE PREFERRED EMBODIMENTS

In the following description, numerous specific details are set forth in order to provide a thorough understanding of the present invention. It will be apparent, however, to one skilled in the art that the present invention may be practiced without some or all of these specific details. In other instances, well known process steps have not been described in detail in order not to unnecessarily obscure the present invention.

Meditation is generally composed of multiple stages. During this whole ‘Guided Meditation’ a soothing ‘ambience sound’ is put in background, which can be silence, rhythmic chanting, music or sounds from nature. For explaining the ‘invention’ with example ‘Guided Meditation’ is divided into following seven stages:

(1) Instructions to begin the ‘Guided Meditation’

(2) Initial prayer

(3) Relaxation based upon breathing or other techniques.

(4) Main ‘Guided Meditation’ based upon a theme.

(5) Recovery cycle to gently bring the person out of the state of Deep relaxation.

(6) Ending prayer

(7) Instructions to end the ‘Guided Meditation’.

During this whole process an ambience is set using the music, rhythmic chanting or sounds from nature.

The ‘Vendor’ or ‘Provider’ will also have the ‘audio data files’ for each of the stages of ‘Guided Meditation’ available in the repository; this will also include various choices for ambience. In addition to that, the ‘audio data files’ can be stored in different languages. This is needed for the case when the customer is not providing some audio data files for the ‘Guided Meditation’ generation.

Each of these phases described here are customizable by the user inputs. On top level the ‘invention’ or ‘method’ works as follows:

(1) Gather the customer inputs and preferences. These preferences can be selection of ‘ambience’, selection of ‘language’, duration of ‘Guided Meditation’, Selection of one or all of the seven individual phases.

(2) Based upon the user inputs, collect the ‘audio files’ for each of the ‘Guided Meditation’ phases and chosen ambience from both the ‘Vendor’ repository and/or from the customer. If the special rhythmic breathing based ‘Guided Meditation’ stage is chosen by the customer and the data is provided then analyze that data using prior art ‘audio processing tools’ and prepare all relevant ‘audio data files’.

(3) Based upon the user selected duration of ‘Guided Meditation’, audio files and the user choices described in (1) generate the ‘assembly instructions’ in the prior art ‘audio processing’ tools language. To generate the ‘Guided Meditation’ of selected duration silence is inserted within or in between the selected ‘Guided Meditation’ stages.

(4) Run the ‘audio processing’ software on these ‘assembly instructions’ to generate a single ‘Guided Meditation’ audio file.

(5) Process this audio file for different formats and audio-media (CD, cassette) and prepare it for delivery.

The above five-step process is described in FIGURE attached at the end of the document.

For step (1) of the ‘method’, the customer may provide with one or some of the following inputs:

Preferences, requirements over Internet/email/phone/postal mail.

If the customer doesn't want to specify preferences and want the software to select the appropriate ‘audio data files’ then the customer is asked questions. Using these questions the background information for the customer is collected, so that a software analysis can be done using that data, to get the ‘Guided Meditation’ that fits customer's personality.

Definition of ‘Guided Meditation’ and stages.

Preferred language for the ‘Guided Meditation’.

Duration of the ‘Guided Meditation’.

Ambience setting during the ‘Guided Meditation’

Optionally provided audio components in industry standard format as audio data files, which can be downloaded over Internet for different stages of ‘Guided Meditation’.

Optionally provided audio components in industry standard format as audio data files on a CD, floppy or a tape, for different stages of ‘Guided Meditation’.

Optionally provided audio components in form of an audiocassette or a CD, for different stages of ‘Guided Meditation’.

‘Audio data files’ for generating special rhythmic breathing ‘Guided Meditation’ stage, which are to be analyzed by the prior art software for patterns of inhale and exhale. Based upon the analysis of these patterns a special ‘Guided Meditation’ stage can be generated. For this special stage, the ‘sound of breathing’ is recorded by the customer under different emotional states. This ‘sound of breathing’ data is used to generate a special ‘Guided Meditation’ stage as explained in next step and further in claims section. For this customer can optionally provide a chant of short duration.

For step (2) all the user provided inputs are analyzed and relevant ‘audio data files’ are gathered or generated. In step (1) an optional special option is to provide a ‘Guided Meditation’ stage that is composed of rhythmic breathing derived out of the customer provided ‘sound of breathing’ audio data file. The ‘Audio data file’ provided by the customer contains the recording of the ‘sound of breathing’ under different emotional states e.g. angry, sad, anxious, depressed etc. This ‘sound of breathing’ is analyzed for inhale and exhale patterns, duration and rhythms using prior art ‘audio processing tools’. The duration and breathing cycle data thus derived is collected for different emotional states of the customer. This duration and breathing cycle data is used to generate a rhythmic breathing ‘Guided Meditation’ stage, if the customer has provided a short duration chant then it can be used to guide the rhythmic breathing, by adjusting the duration of this chant and the interval between two chants.

Also in step (2) based upon the inputs gathered from customer in step (1), the definition of ‘Guided Meditation’ is generated. If the customer is not making any choices then the background information gathered, can be used to find out the ‘Guided meditation’ that is best for the customer. This definition has the meditation stages, duration and their sequence. It also contains the definition of ambience, which will be put into the background for the entire duration of the ‘Guided Meditation’. The ‘ambience’ can be an audio file that covers the entire duration of the ‘Guided Meditation’ or it can be an audio file of small duration that is repeatedly played in a loop. In step (2) all the component audio files are gathered based upon the customer preferences and requirements from both the ‘Vendor’ ‘audio data file’ repository or from the customer. For example if the customer has defined the language for ‘Guided Meditation’ as Spanish then all the ‘audio files’ containing instruction will be chosen from the collection of the ‘audio files’ from ‘Spanish’ language. Some of these files might be provided by the customer as part of input step (1).

In step (3), the ‘Guided Meditation’ definition obtained in step (2) is used to put the ‘audio files’ collected in step (2), in sequence. Based upon the duration of each audio files ‘assembly instructions’ in the ‘audio processing tools’ language are generated. If the customer has defined the duration of the ‘Guided Meditation’ then, it is adjusted as period of silence, within or in between the customer selected ‘Guided Meditation’ phases. The ‘ambience’ is also set either as an ‘audio data file’ covering the full length of the ‘Guided Meditation’, or as an ‘audio data file’ repeated in a loop for the entire duration of the ‘Guided Meditation’. This ambience is also coded as ‘assembly instructions’ in the ‘audio processing tools’ language. The primary function of this step is to generate the ‘assembly instructions’ in the ‘audio processing tools’ language that will generate the complete ‘Guided Meditation’ when executed by the ‘audio processing tool’. To explain the ‘assembly instructions’ we are using a Meditation that has four stages S1, S2, S3 and S4. Each of these stages is contained in prior art industry standard ‘audio data file’ format ‘wav’ as S1.wav, S2.wav, S3.wav, and S4.wav each of duration t1, t2, t3, and t4 respectively. These stages have been put in sequence as S1, S2, S3, and S4. The ambience is provided in ‘audio data file’ A.wav and it is of duration ‘ta’. The user has selected the duration of ‘Guided Meditation’ as ‘T’, which is more, then ‘t1+t2+t3+t4’. Also the customer has chosen to insert the silence after stage S3, to generate a ‘Guided Meditation’ of duration ‘T’. To make the full ‘Guided Meditation’ we provide the tool with control file for each of the stages. These ‘asembly instructions’ can be written in different ways for different ‘audio processing tools’ but for the purpose of explanation of this example we are writing the five control files. Following section describes the syntax of the file containing ‘assembly instruction’. These files are also known as control files.

The ‘Offset’ parameter defines at what absolute time the audio file should be inserted in the full ‘Guided Meditation’. For example the file S1 is to be inserted starting from time=0 in the ‘Guided Meditation’ while file S2 starts from time ‘t1’. In this example period of silence is inserted after end of stage S3, so the file S4 is inserted at time ‘T-t4’ in the ‘Guided Meditation’. This results in a period of silence from time ‘t1+t2+t3’ to ‘T-t4’. The ambience ‘audio data file’ is of duration ‘ta’ which is less then T so it needs to be played multiple times in the background starting at time=0, thus we set the ‘looping’ flag to ‘true’. This ‘looping’ flag will instruct the ‘audio processing tool’ to mix this ambience sound multiple times in loop as a background for the entire duration ‘T’ of the ‘Guided Meditation’. The ‘Source’ parameter tells the tool what input ‘audio data files’ is to be used.

Contents of file S1.ctrl are:

Source=S1.wav

Offest=0

Length=t1

Looping=false

Contents of file S2.ctrl are:

Source=S2.wav

Offest=t1

Length=t2

Looping=false

Contents of file S3.ctrl are:

Source=S3.wav

Offest=‘t1+t2’

Length=t3

Looping=false

Contents of file S4.ctrl are:

Source=S4.wav

Offest=‘T-t4’

Length=t4

Looping=false

Contents of file A.ctrl are:

Source=A.wav

Offest=0

Length=ta

Looping=true

Based upon the ‘assembly instructions’ in the ‘audio processing tools’ language the ‘Guided Meditation’ can be generated either by machine directly if inputs are all submitted directly to machine or by the customer service representative if the inputs are submitted by the email, phone or postal mail.

For example to generate the ‘Guided Meditation’ using the control files from last example, execute following command:
<audio-processing-tool>-i S1.ctrl -i S2.ctrl -i S3.ctrl -i S4.ctrl -o out.wav

In command above the prior art ‘audio processing tool’ takes all the control files as inputs and generate the ‘Guided Meditation’ as an ‘audio data file’ out.wav.

As a last step the generated ‘Guided Meditation’ is processed for delivery. In this step the ‘Guided Meditation’ is converted to industry standard ‘audio formats’ or written down onto some ‘audio data storage media’ like a CD or audiocassette. In this step ‘Guided Meditation’ is also prepared to be delivered by direct Internet download, by email as an audio-data-file attachment.

Although the foregoing invention has been described in some detail for purposes of clarity of understanding, it will be apparent that certain changes and modifications may be practiced within the scope of the appended claims. Accordingly, the present embodiments are to be considered as illustrative and not restrictive, and the invention is not to be limited to the details given herein, but may be modified within the scope and equivalents of the appended claims.

What is claimed is:

1. A method for automatically generating ‘Guided Meditation’ wherein customer selected ‘Guided Meditation’ stages can be used, if specified.

2. A method as recited in claim 1, wherein the customer can further specify language, which can be used to choose the ‘audio data files’ containing instructions in chosen language.

3. A method as recited in claim 2, wherein the customer can further specify the duration of ‘Guided Meditation’, this specified duration is used to calculate the period of silence within or in between the selected ‘Guided Meditation’ stages which results in the ‘Guided Meditation’ of customer specified duration.

4. A method as recited in claim 3, wherein the customer can further select the ambience sound of the ‘Guided Meditation’ or provide an ‘audio data file’ for that, which is mixed in ‘Guided Meditation’ background. Depending upon the duration of the ‘ambience-audio data file’, this can be accomplished by mixing it for the entire duration of the ‘Guided Meditation’ or by putting it in the audio loop.

5. A method as recited in claim 4, wherein the customer can further specify the Meditation Theme, which is used to generate ‘Guided Meditation’ fitting the need of the customer.

6. A method as recited in claim 5, wherein the customer can further answer the questions which are analyzed by prior art software to get customer preferences and requirements, based upon which all suitable and needed ‘audio-data-files’ are chosen from the vendor repository to make a full ‘Guided Meditation’.

7. A method as recited in claim 6, wherein all the customer inputs can be received through email, postal mail, phone or Internet.

8. A method as recited in claim 7, wherein all the customer inputs collected are used to gather the ‘audio-data-files’ from the customer or the vendor repository, to make a complete definition of ‘Guided Meditation’.

9. A method as recited in claim 8, wherein the ‘Guided Meditation’ definition is further refined to have all the stages in a sequence and period of silence inserted within or in between the selected stages to make the ‘Guided Meditation’ of customer specified duration.

10. A method as recited in claim 9, wherein the ‘Guided Meditation’ definition is further refined to have the ambience sound set either for the entire duration of the ‘Guided Meditation’ or as an audio loop that repeats in the background.

11. A method as recited in claim 10, wherein the definition of ‘Guided Meditation’, is used to generate the ‘assembly instructions’ in prior art ‘audio processing tools’ language.

12. A method as recited in claim 11, wherein the ‘assembly instructions’ are executed by the prior art ‘audio processing tools’ on a computer either directly or by the customer service representative to generate the complete ‘Guided Meditation’.

13. A method as recited in claims 12, wherein the generated ‘Guided Meditation’, is processed as an audio data file, which can be downloaded over the Internet or can be sent as an email attachment.

14. A method as recited in claims 13, wherein the generated ‘Guided Meditation’ is written on audio media like audiocassette or CD, which can be mailed to the customer.

15. A method for automatically generating ‘Guided Meditation’ in which one stage is defined by rhythmic breathing, for which the ‘sound-of-breathing audio data’ is provided as an ‘audio data file’ by the customer.

16. A method as recited in claim 15, further comprising of the ‘sound-of-breathing audio data’ as provided by the customer, which was recorded during different emotional states of the customer (angry, sad, anxious, depressed etc.) by the customer.

17. A method as recited in claim 16, further comprising of the customer specified audio file containing a chant of a specified small duration. This chant will be used to guide the rhythmic breathing during this special stage of ‘Guided Meditation’ by altering its duration and frequency of repetition.

18. A method as recited in claim 17, wherein the ‘sound-of-breathing data’ corresponding to different emotional states of the customer, is further analyzed by prior art software for inhale and exhale pattern, duration and rhythm.

19. A method as recited in claim 18, wherein a rhythmic chant is generated corresponding to the analyzed inhale and exhale pattern, duration and rhythm. The pattern, duration and rhythm of the chant is altered to match the analyzed inhale and exhale pattern, duration and rhythm. This is done for each ‘sound-of-breathing audio data’ data file corresponding to each emotion specified by customer.

20. A method as recited in claim 19, wherein the special ‘Guided Meditation’ stage is generated as an audio data file, using the sequences of the rhythmic chanting corresponding to each emotional state specified by customer.
